(19) United States
(12) Patent Application Publication
(10) Pub. No.: US 2005/0228304 A1
(43) Pub. Date: Oct. 13, 2005

(54) Method and system of respiratory therapy employing heart rate variability coherence

(76) Inventor: Stephen Bennett Elliott, Allen, TX (US)

Correspondence Address: Stephen Bennett Elliott, 702 Buffalo Springs Drive, Allen, TX, 75013, US

(21) Appl. No.: 10/814,035

(22) Filed: Apr. 1, 2004

(51) Int. Cl. A61M 15/00; A61M 16/10; A61B 5/02; A61B 5/08

(52) U.S. Cl. 600/508; 128/905; 600/529

(57) ABSTRACT

The present invention provides a broad method and system for bringing the heart rate variability cycle of a respiratory care recipient to the desirable state of coherence thereby bringing cardiopulmonary functioning of the therapy recipient to the optimal level of functioning, during which respiratory therapy is provided, yielding maximal effectiveness and efficiency of said care. An instructive method in the application of the preferred embodiments is provided for respiratory therapy practitioners and therapy recipients.

### RELATED PATENT FILINGS

Method and System for Consciously Synchronizing the Breathing Cycle with the Natural Heart Rate Cycle (Ser. No. 10/699,025), System and Method for Synchronizing the Heart Rate Variability Cycle With The Breathing Cycle (Feb. 19, 2004), Method of Presenting Audible and Visual Cues for Synchronizing the Breathing Cycle With An External Timing Reference for Purposes of Synchronizing The Heart Rate Variability Cycle With The Breathing Cycle (Mar. 15, 2004), Method and System Providing A Fundamental Musical Interval for Heart Rate Variability Synchronization (Mar. 23, 2004)

### FIELD OF THE INVENTION

The present invention relates to the field of medicine and in particular to the field of respiratory therapy. More particularly, it relates to the field of respiratory therapy wherein a human subject breathes air or other gases that are enriched with oxygen or other additives for the purpose of regaining, sustaining, or enhancing health.

### BACKGROUND OF THE INVENTION

A primary aspect of the field of respiratory therapy involves the application of oxygen or other gases for purposes of improving, sustaining, or enhancing health. These gases may be of a pure form or they may contain additives in the form of the various medications that may be delivered via the mechanism of breathing.

This form of respiratory therapy may be applied in two fundamental ways depending on the state of consciousness of the subject. When the subject is unconscious, respiratory therapy must be applied without conscious participation. Alternatively, when the subject is conscious and participative, therapy may be applied with the subject's conscious participation. The present invention applies to the latter case, that is, the case wherein there is conscious participation of the care recipient.

It is known that cardiopulmonary efficiency is greatest when the heart and lungs are working in synchrony, this synchrony being identified by the physiological state known as “coherence” of the heart rate variability cycle. In fact, a high degree of coherence of the heart rate variability cycle cannot exist without synchrony between the heart rate variability cycle and the breathing cycle. This synchrony is characterized by the inhalation phase of breathing occurring coincident with increasing heart rate and the exhalation phase of breathing occurring coincident with decreasing heart rate.

While the heart has its own tendency toward rhythm, it is closely coupled to the rhythm of the breathing cycle. The relationship is such that as inhalation occurs, the heartbeat rate tends to increase and as exhalation occurs, the heartbeat rate tends to decrease. It is important to note that while the heartbeat rate and breathing rate influence each other, the relationship is a plesiochronous one, that is, they are independent rhythms that strongly influence but do not directly govern each other. Consequently, it is possible for a human subject to be inhaling while their heart rate is decreasing and exhaling while their heart rate is increasing. This is an undesirable condition and inconsistent with the natural cardiopulmonary functioning of the body.

Alternatively, it is natural and desirable to inhale while the heart rate is increasing and exhale while the heart rate is decreasing. This is consistent with the natural cardiopulmonary operation of the body, functions of which are to extract gases from the air, place them into the bloodstream, and circulate them throughout the body where they may be utilized for metabolic processes, the absorption function being maximized when inhalation and increasing heart rate are synchronized. Similarly, the function of exchanging carbon dioxide and other gaseous waste products from the bloodstream and expelling them through the lungs is maximized when exhalation and decreasing heart rate are synchronized.

The present invention takes advantage of the relationship between the breathing cycle and the natural heart rate variability cycle to bring the heart rate variability cycle to the desired state of coherence, and consequently, the cardio-pulmonary system to its maximal efficiency and effectiveness. In this state of maximal efficiency and effectiveness, respiratory therapy may be applied with the following advantages:
1) optimal transfer and absorption of gases into the bloodstream,
2) optimal circulation of gases throughout the body,
3) optimal exchange of gaseous waste products, that is, carbon dioxide, etc.,
4) consequent increase in therapeutic efficacy,
5) consequent decreases in time of treatment and duration of therapy,
6) consequent reduction in the volume of gases and or medications applied over the course of treatment with consequent reductions in the cost of therapy.

### SUMMARY OF THE INVENTION

The present invention applies prior inventions including Method and System for Consciously Synchronizing the Breathing Cycle with the Natural Heart Rate Cycle (Ser. No. 10/699,025) and System and Method for Synchronizing the Heart Rate Variability Cycle with the Breathing Cycle (February, 2004) specifying methods and systems for achieving coherence of heart rate variability by either consciously synchronizing the breathing cycle with the heart rate variability cycle or by facilitating synchronization of the heart rate variability cycle with the breathing cycle. Additionally, it applies systems and methods of prior inventions including Method of Presenting Audible and Visual Cues for Synchronizing the Heart Rate Variability Cycle With An External Timing Reference for Purposes of Synchronizing the Heart Rate Variability Cycle with the Breathing Cycle (Mar. 15, 2004), and Method and System Providing A Fundamental Musical Interval for Heart Rate Variability Synchronization (Mar. 23, 2004) to provide audible, visual, and tactile indicators for purposes of indicating to a conscious respiratory therapy recipient, when to inhale and when to exhale, thereby maintaining the state of coherence of the heart rate variability cycle while the recipient undergoes respiratory therapy in the form of breathing therapeutic gases and or gas-borne medications.

A second preferred embodiment of the present invention provides the method and system for regulating the flow of gases during the application of respiratory therapy in accordance with the breathing cycle.

Finally, an instructive method is specified for both respiratory therapy practitioners and care recipients in the application of the preferred embodiments of the present invention.

### BRIEF DESCRIPTION OF THE DRAWING FIGURES

The accompanying drawing figures incorporated in and forming a part of this specification illustrate several aspects of the invention and together with the description serve to explain the principles of the invention.

FIG. 1 depicts the application of respiratory therapy while the heart rate is being monitored and the recipient is consciously synchronizing their breathing cycle with their heart rate variability cycle.

FIG. 2 depicts the application of respiratory therapy while the recipient is synchronizing their breathing cycle to an external timing reference and their heart rate variability is not being monitored.

FIG. 3 depicts a programmable regulator operating on the basis of the heart rate variability cycle.

FIG. 4 depicts a programmable regulator operating on the basis of a timing reference.

FIG. 5 provides a detailed description of the electrically controlled regulator referenced in FIGS. 1 though 4.

FIG. 6 presents a table describing the operation of the electrically controlled regulator.

### DETAILED DESCRIPTION OF THE PREFERRED EMBODIMENTS

The present invention provides a method and system by which respiratory therapy may be optimally applied to conscious recipients by bringing the recipient's heart rate variability cycle into the desired state of coherence, thereby bringing their cardiopulmonary system into optimal efficiency and effectiveness. This is accomplished in two principal ways. Firstly, by applying respiratory therapy while monitoring the recipients heart rate variability cycle and instructing the recipient to consciously synchronize their breathing cycle with their heart rate variability cycle per prior invention, Method and System for Consciously Synchronizing the Breathing Cycle with the Natural Heart Rate Variability Cycle (Ser. No. 10.699,025). Secondly, by applying respiratory therapy while instructing the recipient to synchronize their breathing cycle with an external timing reference with a fundamental periodicity of 11.8 seconds per prior invention, System and Method for Synchronizing the Heart Rate Variability Cycle With The Breathing Cycle. The detailed description of preferred embodiments will now commence.

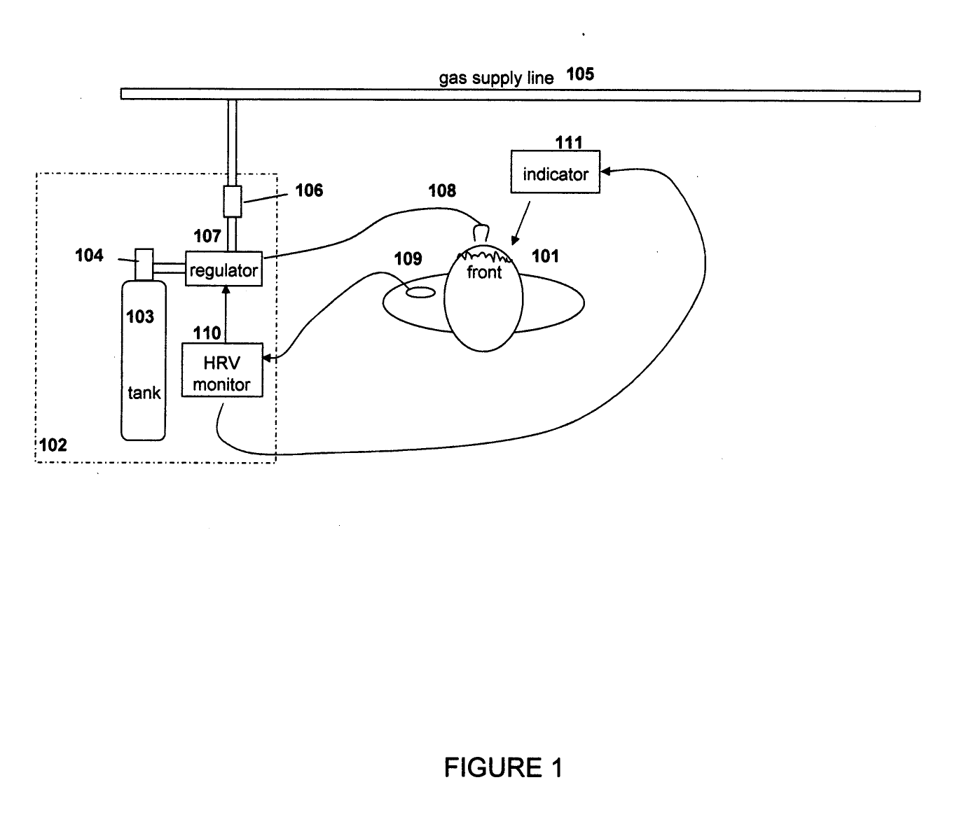

Referring to FIG. 1, respiratory therapy recipient 101 is in either a seated or reclining position. Gas delivery system 102 typically consists of a tank 103 or supply line 105 from which gas is delivered in the location of the recipient. This may be a portable delivery system or an integral part of a health care physical infrastructure as may be found in a hospital. Tank 103 or supply line 105 are buffered by primary regulator/check valves 104 and 106, respectively. Gas delivery system 102 may or may not provide the facility for local addition and mixing of other therapeutic gases or medications. Electrically controlled regulator 107 facilitates the automatic control of gas delivery. Cannula 108 is fitted to the recipient and connected to electrically controlled regulator 107. During therapy, gases flow from tank 103 or supply line 105 through primary regulators 104 or 106, through electrically controlled regulator 107, and through cannula 108 to recipient 101.

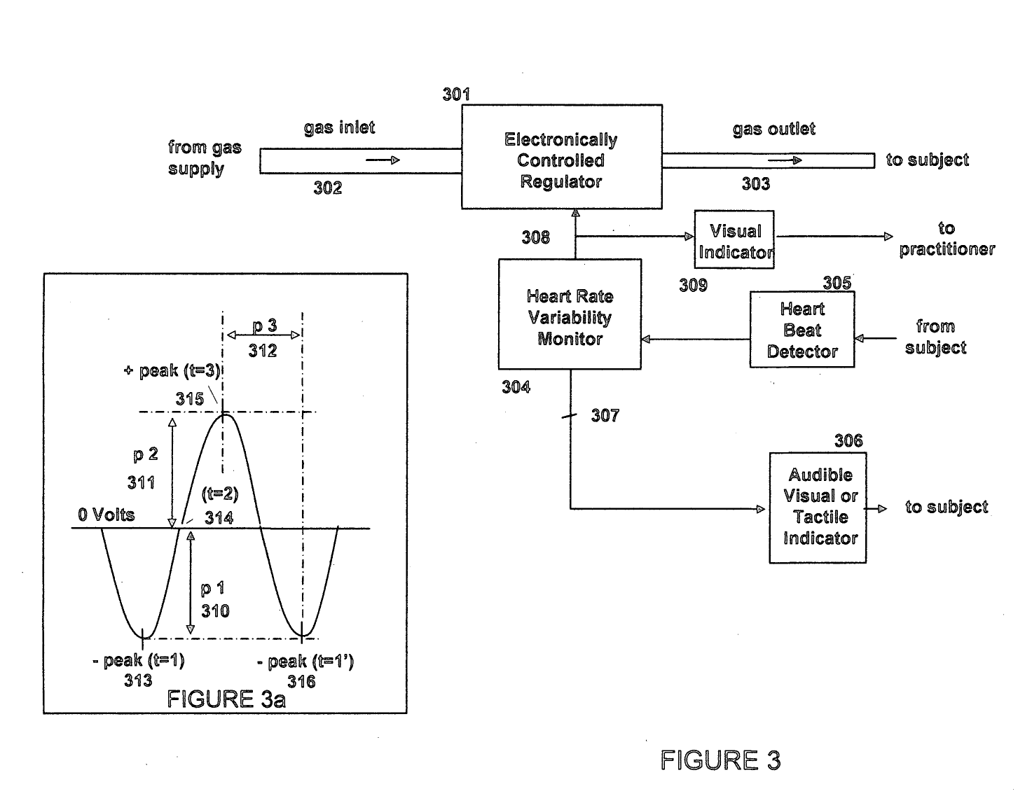

Heart rate detector 109 is fitted to recipient 101 such that the heart rate variability cycle can be monitored during the application of respiratory therapy. Heart rate detector 109 is attached to heart rate variability monitor 110 where positive going and negative going phases are discriminated per prior invention Method and System for Consciously Synchronizing the Breathing Cycle with the Natural Heart Rate Cycle (Ser. No. 10/699,025). A positive going phase is the interval between the lowest heart beat rate in beats per second and the highest heart rate in beats per second. A negative going phase is the interval between the highest heart beat rate in beats per second and the lowest heart beat rate in beats per second. These positive and negative phases are presented to subject 101 via transducer 111 in either audible, visual, or tactile format depending on what form is most applicable to the needs of subject 101. Subject 101 is instructed to synchronize his inhalation with positive going phases and his exhalation with negative going phases. Per a preferred embodiment of the present invention, a separate output of heart rate variability monitor 110 connects to electrically controlled regulator 107 for the purpose of synchronizing regulator operation with the heart rate variability cycle of the subject. In this way, the subject's breathing cycle is synchronized with their heart rate variability cycle bringing the subjects cardiopulmonary system into optimal efficiency and effectiveness during which respiratory therapy is applied. Gas flows to the subject during the inhalation phase of the breathing cycle and is stopped during the exhalation phase of the breathing cycle. The function of controlling electrically controlled regulator 107 will be explained in more detail in FIG. 3.

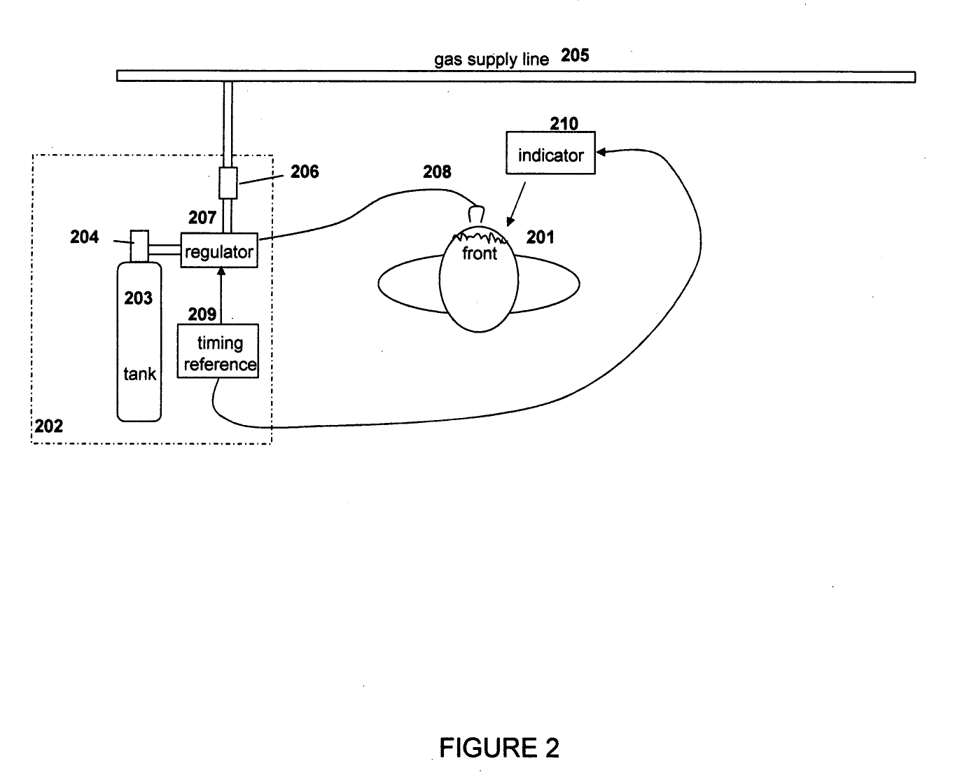

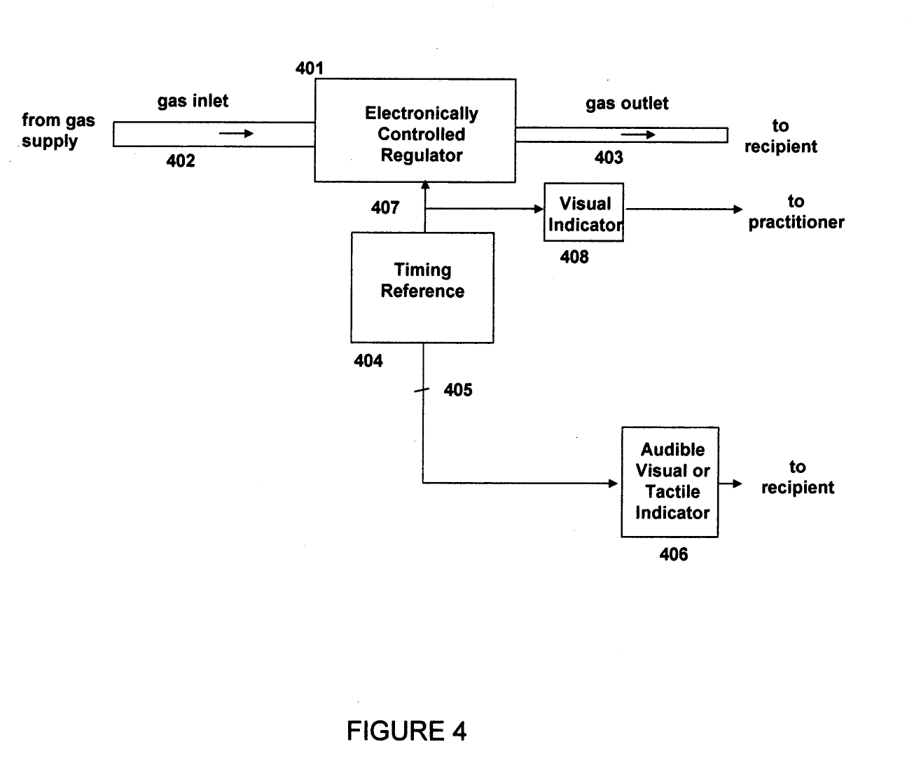

FIG. 2 applies prior inventions System and Method for Synchronizing the Heart Rate Variability Cycle With The Breathing Cycle (February 2004), Method of Presenting Audible and Visual Cues for Synchronizing the Breathing Cycle With An External Timing Reference for Purposes of Synchronizing The Heart Rate Variability Cycle With The Breathing Cycle (Mar. 15, 2004), and Method and System Providing A Fundamental Musical Interval for Heart Rate Variability Synchronization (Mar. 23, 2004). In the case of FIG. 2 and per prior invention, System and Method for Synchronizing the Heart Rate Variability Cycle With The Breathing Cycle (February 2004), the subject synchronizes their breathing cycle with a timing reference signal with a fundamental periodicity of 11.8 seconds. The subject is instructed to inhale on positive going phases of the 11.8 second interval, the positive phase equaling 5.9 seconds, and exhale on negative going phases of the 11.8 second interval, the negative going phase equaling 5.9 seconds. These phases are presented to subject 201 via indicator 210 which may be of an audible, visual, or tactile variety depending on what is appropriate. When the subject follows this method, their heart rate variability cycle synchronizes with their breathing cycle bringing the cardio-pulmonary system into optimal efficiency and effectiveness during which respiratory therapy is applied. A separate output from timing reference 209 connects to electrically controlled regulator 207 for the purpose of synchronizing regulator operation with the timing reference and thereby synchronizing the operation of electrically controlled regulator 207 with the breathing cycle of the subject. Gas flows to the subject during the inhalation phase of the breathing cycle and is stopped during the exhalation phase of the breathing cycle. The function of controlling electrically controlled regulator 207 will be explained in more detail in FIG. 4.

FIG. 3 provides a more in depth discussion of concepts discussed relative to FIG. 1. FIG. 3 describes the preferred embodiment of the present invention wherein previously mentioned electrically controlled regulator 107 is controlled. Referring to FIG. 3, the objective of this control is to open programmable regulator 301 during the inhalation phase of breathing and close programmable regulator 301 during the exhalation phase of breathing thereby reducing the volume of gases and medications and consequent cost by 50%. Gas is delivered to electrically controlled regulator 301 via gas inlet 302. Gas exits regulator 301 and is delivered to the recipient via gas outlet 303 under control of regulator 301. The throughput of regulator 301 is controlled by heart rate variability monitor 304 via connector 308. Per prior invention, Method and System for Consciously Synchronizing the Breathing Cycle with the Natural Heart Rate Variability Cycle (Ser. No. 10/699,025), a function of heart rate variability monitor 304 is to monitor the heart beat rate and detect positive and negative going phases of the heart rate cycle so as to present those phases to the subject for the purpose of synchronizing the breathing cycle with the heart rate variability cycle. Information pertaining to heart rate variability phase is presented via connector 307 to the subject via audible, visual, or tactile indicator 306. As previously mentioned, per the present invention, a second heart rate variability monitor output 308 is provided for the purpose of synchronizing operation of electrically controlled regulator 301 with the heart rate variability cycle of the subject. Given that the subject's breathing cycle is synchronized with their heart rate variability cycle, per the instruction given the subject, regulator 301 opens during the inhalation phase of breathing and closes during the exhalation phase of breathing. This function is explained in more detail in FIG. 3a.

FIG. 3a provides a graphical representation of heart beat rate as seen by heart rate variability monitor 304 as provided by heart beat detector 305. Per the present invention, a function of heart rate variability monitor 304 is to discriminate between positive and negative phases of the heart rate variability cycle, the positive phase defined by the interval between peak negative heart beat rate 313 and peak positive heart beat rate 315, and the negative phase defined by the interval between peak positive heart beat rate 315 and peak negative heart beat rate 316, for the purpose of controlling electrically controlled regulator 301. Three periods are critical to realizing the required control. During period 1 310, the regulator transitions from being completely closed to being completely open. During period 2 311, the regulator transitions from being completely open to being completely closed. During period 3 312, the regulator remains closed. In this way, the flow of gas through electrically controlled regulator 301 is synchronized with the inhalation of the subject such that peak flow occurs at mid-inhalation. Note that when a cannula is employed, the subject remains able to inhale regardless of whether or not gas is flowing. Visual indicator 309 is indicative of the degree to which electrically controlled regulator 301 is open and consequently, is indicative of gas flow.

FIG. 4 provides a more in depth discussion of concepts relative to FIG. 2. FIG. 4 describes the preferred embodiment of the present invention wherein previously mentioned electrically controlled regulator 207 is controlled. Again, FIG. 4 differs from FIG. 3 in the same way that FIG. 2 differs from FIG. 1, that being the absence of heart rate variability monitor and heart beat detector 304 and 305, respectively, as depicted in FIG. 3, and the addition of timing reference 404 as depicted in FIG. 4. As previously mentioned relative to FIG. 2, FIG. 4 applies prior inventions, System and Method for Synchronizing the Heart Rate Variability Cycle with The Breathing Cycle (February 2004), Method of Presenting Audible and Visual Cues for Synchronizing the Breathing Cycle With An External Timing Reference for Purposes of Synchronizing The Heart Rate Variability Cycle With The Breathing Cycle (Mar. 15, 2004), and Method and System Providing A Fundamental Musical Interval for Heart Rate Variability Synchronization (Filed Mar. 23, 2004). In this case, timing reference 404 provides an output 405, representative of the breathing cycle such that inhalation and exhalation phases can be communicated to the subject via audible, visual, or tactile indicator 406.

Per the present invention, timing reference 404 provides a second output 407, for purposes of controlling electrically controlled regulator 401. This control is essentially the same as that described relative to FIG. 3, the objective being to synchronize the flow of gas through electrically controlled regulator 401 with the breathing cycle of subject. Timing requirements relative to this function are identical to those of FIG. 3a. Visual indicator 408 is indicative of the degree to which electrically controlled regulator 401 is open, and consequently, is indicative of gas flow.

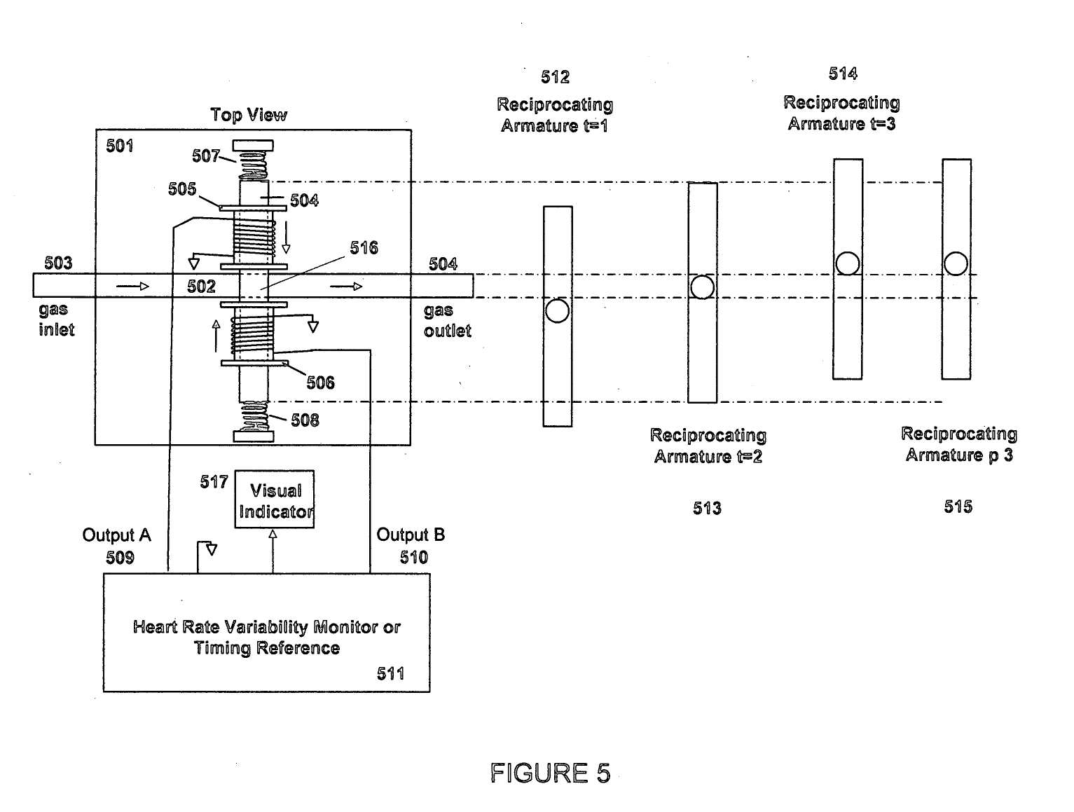

FIG. 5 presents a more detailed view of electrically controlled regulators 301 and 401 which are of identical design. Per FIG. 5, the primary function of electrically controlled regulator 501 is to synchronize the delivery of gas with the breathing cycle of the recipient. To accomplish this, regulator 501 must open as the inhalation phase begins, achieving maximal flow at mid-inhalation, and begin closing such that it is fully closed at the end of inhalation. It must then remain closed for the duration of exhalation. This function is accomplished by a reciprocating armature 504 that reciprocates under control of the heart rate variability monitor or timing reference 511. This is accomplished by alternately driving independent coils 505 and 506 which are wound in opposition such that 505 serves to move the armature downward and 506 serves to move the armature upwards. When neither coil is activated springs 507 and 508 counterbalance each other such that the armature rests in the center, maximally opening the channel through which gas flows from inlet 503 to outlet 504. This is the resting position of the armature when heart rate variability monitor or timing reference 511 is not turned on or not provided.

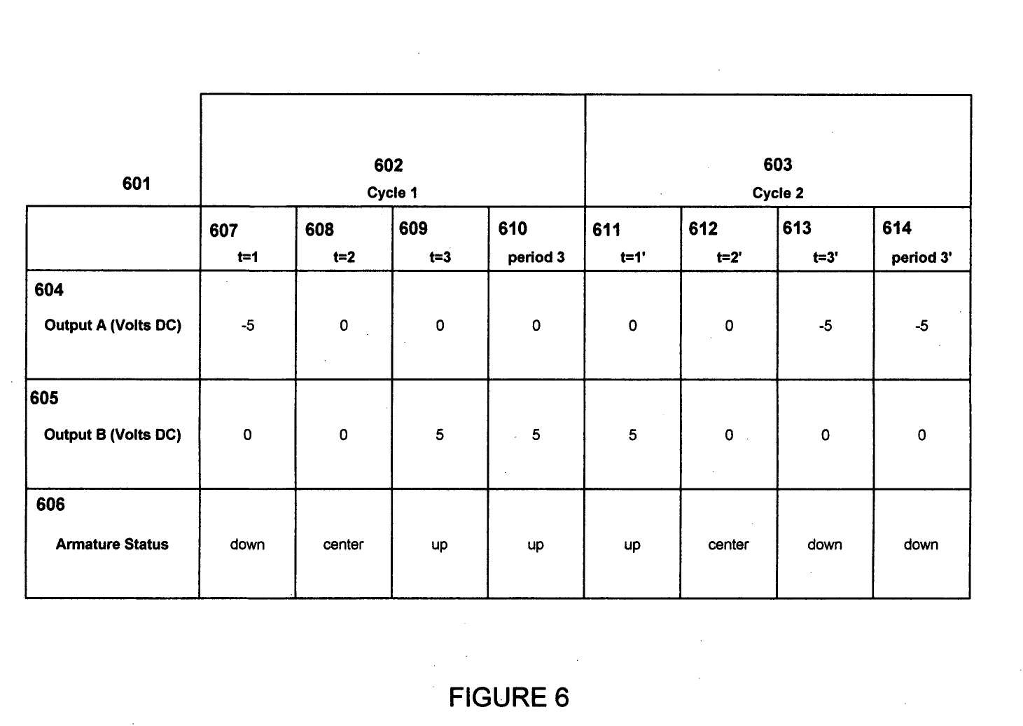

A more detailed discussion of the cyclic functioning of electrically controlled regulator 501 will now commence. 512, 513, 514, and 515 present the status of reciprocating armature 504 at different moments of one cycle of operation as identified in FIG. 3a, t=1 313, t=2 314, t=3 315, and p 3 312. This functioning is further elucidated in FIG. 6 table 601 wherein 2 consecutive cycles of operation are described. Referring back to FIG. 5, at the beginning of cycle 1, as characterized by moment t=1 313 of FIG. 3, output A 509 of heart rate variability monitor or timing reference 511 is negative 5 volts, the peak negative output corresponding to the lowest heart beat rate and the end of the exhalation phase of the breathing cycle. This negative 5 volt output actuates coil 505, driving reciprocating armature 504 to its downward most position 512 closing valve 516. Therefore at t=1 313, valve 516 is fully closed. At t=2 314, output A 509 of heart rate variability monitor or timing reference 511 has transitioned from negative 5 volts to 0 volts. It is important to note that while voltages are specified at particular moments, the outputs of heart rate variability monitor or timing reference 511 are analog voltages representing the sinusoidal nature of the heart rate variability and breathing cycles. With this transition, reciprocating armature 504 moves from its downward most position 512 to center position 513, aligning the hole with channel 502 thereby maximally opening valve 516. At t=2 314, output B 510 of heart rate variability monitor or timing reference 511, heretofore at 0 volts, begins to transition from 0 volts to positive 5 volts, actuating coil 506 and driving reciprocating armature 504 in the upward direction. As this happens, valve 516 begins to close. At t=3 315, output B 510 has reached its peak output of positive 5 volts driving armature 504 to its upward most position per 514. During the entirety of period 3, p 3 312, armature 504 remains in its upward most position 515, fully closing valve 516. Visual indicator 517 is indicative of gas flow through valve 516 and varies such that it is brightest when outputs A 509 and B 510 are zero volts. At t=1′ 316 this cycle repeats itself in a reverse order. This order is fully described in FIG. 6 wherein two complete cycles of operation are described. The process outlined in table 601 repeats itself for the duration of the therapy session. This completes the discussion of FIGS. 1-6.

An instructive method is also specified for use by respiratory care practitioners and care recipients. Two different instructive methods are provided depending on which preferred embodiment is being employed, that employing monitoring of the heart rate, or that employing a timing reference.

Instructive Method Employing Heart Rate Monitoring:

1. The care recipient is instructed to assume a comfortable posture and relax as deeply as possible.
2. The care practitioner applies a heart rate monitor to the care recipient.
3. The care practitioner selects the mode of indication that is most suitable to the care recipient.
4. The care practitioner applies a cannula to the care recipient, turns on, and adjusts the supply of gas at the primary regulator.
5. Care recipients are instructed to pay attention to the indicator, inhaling when and for the duration indicated, and exhaling when and for the duration indicated.
6. The care practitioner observes the care recipient and the gas delivery visual indicator to assess the degree of synchronization that is occurring between the recipient's breathing and gas delivery.
7. The care practitioner councils the care recipient in achieving the desirable degree of synchronization.
8. The care practitioner instructs the care recipient to continue breathing in the synchronized manner for the duration of therapy.
   Instructive Method Employing A Timing Reference:
1. The care recipient is instructed to assume a comfortable posture and relax as deeply as possible.
2. The care practitioner selects the mode of indication that is most suitable to the care recipient.
3. The care practitioner applies a cannula to the care recipient, turns on, and adjusts the supply of gas at the primary regulator.
4. The care practitioner turns on the timing reference.
5. Care recipients are instructed to pay attention to the indicator, inhaling when and for the duration indicated, and exhaling when and for the duration indicated.
6. The care practitioner observes the care recipient and the gas delivery visual indicator to assess the degree of synchronization that is occurring between breathing of the care recipient and gas delivery.
7. The care practitioner councils the care recipient in achieving the desirable degree of synchronization.
8. The care practitioner instructs the care recipient to continue breathing in the synchronized manner for the duration of therapy.

Those skilled in the art will recognize improvements and modifications to the preferred embodiments of the present invention. All such improvements and modifications are considered within the scope of the concepts disclosed herein and the claims that follow.

1. The broad method of respiratory therapy wherein the care recipient's breathing cycle is consciously synchronized with their heart rate variability cycle for purposes of achieving coherence of the their heart rate variability cycle for purposes of achieving optimal cardiopulmonary functioning and consequent optimal therapeutic efficacy and efficiency.

2. The broad method of respiratory therapy wherein synchronization of the care recipient's breathing cycle with an external timing reference is achieved for purposes of synchronizing their heart rate variability cycle with their breathing cycle for purposes of achieving coherence of their heart rate variability cycle for purposes of achieving optimal cardio-pulmonary functioning and consequent optimal therapeutic efficacy and efficiency.

3. The broad system of claim 1 wherein synchronization of the care recipient's breathing cycle with their heart rate variability cycle is achieved for purposes of achieving optimal cardiopulmonary functioning and consequent optimal therapeutic efficacy and efficiency.

4. The broad system of claim 2 wherein synchronization of the care recipient's breathing cycle with an external timing reference is achieved for purposes of synchronizing their heart rate variability cycle with their breathing cycle for purposes of achieving coherence of their heart rate variability cycle for purposes of achieving optimal cardiopulmonary functioning and consequent optimal therapeutic efficacy and efficiency.

5. The specific method of claims 1 and 2 wherein gas flow is synchronized with the heart rate variability signal of the care recipient or an external timing reference such that gas is delivered during the inhalation phase of the breathing cycle and is suspended during the exhalation phase of the breathing cycle thereby reducing gas consumption.

6. The specific system of claim 5 wherein gas flow is determined by an electrically controlled regulator that is controlled by a heart rate variability monitor, or alternatively, a timing reference.

7. The specific system of claim 6 wherein a reciprocating armature forms the basis of the electrically controlled regulator and is controlled such that its operation and consequent gas flow through the electrically controlled regulator is synchronized with the breathing cycle of the care recipient.

8. The system of claim 7 wherein the reciprocating arm of the electrically controlled regulator rests at its center point when heart rate variability monitor or timing reference are powered down or removed from the system, thereby facilitating maximal flow of gas through the system.

9. The specific system of claim 5 wherein a visual indicator representing the degree of gas flow is provided to the respiratory care practitioner for purposes of gauging the synchrony of the recipient's breathing cycle with the deliver of gas.

10. The specific system of claim 6 wherein the timing and logic for controlling electrically controlled regulator is specified.

11. The instructive method of claim 1 wherein a respiratory care practitioner and or care recipient is instructed in the use of the preferred embodiment of the invention employing the monitoring of heart rate variability.

12. The instructive method of claim 2 wherein a respiratory care practitioner and or care recipient is instructed in the use of the preferred embodiment of the invention employing a timing reference.
